(19) United States
(12) Patent Application Publication
(10) Pub. No.: US 2011/0151479 A1
(43) Pub. Date: Jun. 23, 2011

(54) MICROFLUIDIC SYSTEMS INCORPORATING FLOW-THROUGH MEMBRANES

(75) Inventors: Dean Y. Stevens, Seattle, WA (US); Lisa K. Lafleur, Kirkland, WA (US); Berry R. Lutz, Seattle, WA (US); Paolo Spicar-Mihalic, Seattle, WA (US); Paul Yager, Seattle, WA (US)

(73) Assignee: University of Washington, Seattle, WA (US)

(21) Appl. No.: 13/060,664

(22) PCT Filed: Aug. 25, 2009
(86) PCT No.: PCT/US09/54941

§ 371 Date: Feb. 24, 2011

Related U.S. Application Data
(60) Provisional application No. 61/091,639, filed on Aug. 25, 2008.

(51) Int. Cl. G01N 33/53; G01N 33/48; G01N 30/00; G01N 1/00

(52) U.S. Cl. 435/7.1; 422/68.1; 422/69; 436/174

(57) ABSTRACT

Disclosed is a flow-through membrane assay that takes advantage of a high surface area and rapid transport while allowing individual control over flow rates and times for each step of a multi-step assay. A microfluidic card features channels in communication with a porous membrane, channels on either side of membrane to allow transverse flow across the membrane, capturing a labeled target from the sample by flowing the sample across the membrane, or capturing a target from the sample followed by flowing a reagent containing a label that binds to the target. Fluid can be pushed or pulled through the assay membrane by external control. Air near the membrane is managed by diverting air between fluids to a channel upstream of the assay membrane, venting air between fluids through a hydrophobic membrane upstream of the assay membrane, and/or by venting trapped air through a hydrophobic membrane downstream of the assay membrane.

This application claims benefit of U.S. provisional patent application No. 61/091,639, filed Aug. 25, 2008, the entire contents of which are incorporated herein by reference. This application is related to PCT application number US07/80479, filed Oct. 4, 2007, the entire contents of which are incorporated herein by reference.

### TECHNICAL FIELD OF THE INVENTION

This invention relates generally to methods and devices using porous flow-through membranes in molecular affinity assays performed in a microfluidic environment. The invention relates to use of such membranes for a variety of operations, including filtering, solid-phase assay and selective capture.

### BACKGROUND OF THE INVENTION

Immunoassays take advantage of the specific binding abilities of antibodies to be widely used in selective and sensitive measurement of small and large molecular analytes in complex samples. The driving force behind developing new immunological assays is the constant need for simpler, more rapid, and less expensive ways to analyze the components of complex sample mixtures. Current uses of immunoassays include therapeutic drug monitoring, screening for disease or infection with molecular markers, screening for toxic substances and illicit drugs, and monitoring for environmental contaminants.

Porous membranes are used in conventional lateral flow and flow-through cartridges, in which flow of fluid occurs by wicking through the membrane (either laterally or transverse) into an absorbent pad. The dependence on wicking to generate flow greatly limits the control over assay conditions. Previously published patents using membranes for immunoassays are largely in the area of lateral flow and flow-through by wicking. Examples of patents describing assays using flow-through by wicking include: U.S. Pat. Nos. 4,632,901 & 4,727,019 to Valkirs; 4,861,711 to Friesen; 5,079,142 to Coleman; and 7,300,802 to Paek. Examples of flow-through by wicking, in a card-based format, include U.S. Pat. Nos. 5,369,007 to Kidwell, and 6,663,833 to Stave. An example of an on-card membrane assay is provided in U.S. Pat. No. 6,303,389 to Levin (cassette design only).

All of the lateral flow assays are essentially limited to a single step in which sample (and buffer) is added to the sample pad, and it flows by capillary action (wicking) along the pad. For the single step methods, the sample is premixed with the detection label as it flows through a storage pad, and the complex binds to the capture region. This premixing leads to false negatives at high analyte concentrations (the “hook effect”), and it does not allow individual control over binding reactions that are normally optimized individually in bench top assays to improve performance. The flow-through formats normally allow different reagents to be added in sequence, but without control over the flow rates of reagents. None of the prior art describes a microfluidic system that performs a detection assay by flowing fluid through a porous membrane using a controllable external force (e.g., pumping, pressure, vacuum, gravity).

There remains a need for controlling assay conditions, particularly fluids, in microfluidic devices with flow-through membranes. The invention described herein meets these needs and more through the application of external force to regulate the flow of fluid transversely through the assay membrane.

### SUMMARY OF THE INVENTION

The invention provides an assay device and methods for detection of an analyte in a fluidic sample. The device comprises a microfluidic chamber having first and second channels. The first channel is defined by walls and a floor, the channel having an upstream end and a downstream end, wherein fluid brought into contact with the channel flows from the upstream end toward the downstream. The floor comprises a region between the upstream and downstream ends that contains a porous membrane having an upper surface and a lower surface. The second channel is defined by walls and a ceiling, wherein the ceiling comprises the lower surface of the porous membrane. The device further comprises one or more capture agents immobilized on the porous membrane, and means for regulating the flow of fluid transversely through the porous membrane, across the upper surface and the lower surface, via application of an external force within the first and/or second channel. The device permits detection of analyte captured on the porous membrane in a rapid, accurate and controlled manner.

The regulation of fluid flow across the porous membrane allows for multi-step assays with individual control over flow rates and times for each step of the assay. For example, incubation with sample, reagents (such as secondary antibodies, enzyme substrates) and washes can each be separately tuned. This regulation is achieved through use of a controllable, external force. In one embodiment, the means for regulating the flow of fluid comprises a pneumatic device, a pump, a valve, or a change in gravitational force or static head, such as by altering the planar orientation of the device or releasing fluid driven toward the porous membrane otherwise stopped by a valve. The pneumatic device can comprise, for example, a pump or a vacuum.

The assay device can further comprise a hydrophobic membrane disposed within the first channel. A hydrophobic membrane can be used to selectively remove air (rather than water) from the channel. The hydrophobic membrane can be disposed within a wall that communicates with the atmosphere or with another chamber that comprises a vacuum or other means to remove air from the channel.

A waste channel, with or without a hydrophobic membrane, can also be disposed within the first and/or second channel to provide an outlet for removal of air or other unwanted material. The removal of air from the fluid stream prevents blockage of the membrane, as wet membranes are impermeable to air. This removal of air that would otherwise be in contact with the membrane contributes to the regulation of fluid flow across the assay membrane. This air removal allows fluids to access the membrane and facilitates the delivery of upstream fluid to the membrane.

In some embodiments, the assay device further comprises a reagent storage depot in communication with the first channel, and a plurality of detection reagents disposed within the storage depot. In some embodiments, the reagent storage depot comprises a porous material. In other embodiments, the reagent storage depot comprises a sealed chamber that releases the detection reagents into the first channel upon rupture of the sealed chamber. The assay device can further comprise means for detecting analyte bound to the capture agent on the porous membrane.

The invention further provides a method for detection of an analyte in a fluidic sample. The method comprises contacting the fluidic sample with the porous membrane of the assay device of the invention and contacting a fluid containing a reagent with the porous membrane. The method further comprises regulating the flow of fluid transversely through the porous membrane across the upper surface and the lower surface via application of an external force within the first and/or second channel; and detecting the presence of analyte bound to reagent on the porous membrane.

The contacting of a fluid containing a reagent with the porous membrane can comprise contacting a fluid with a reagent storage depot disposed within the assay device, wherein the reagent is stored in the storage depot in anhydrous form and is mobilized upon contact with the fluid. Alternatively, the contacting of can comprise rupturing a reagent storage depot disposed within the assay device, wherein the reagent is stored in the storage depot and is mobilized upon rupture of the reagent storage depot. For example, a movable pin or other sharp implement can be disposed within the device such that actuation of the pin, e.g., by squeezing or other motion, moves the pin into place to rupture the reagent store depot. In such an embodiment, the reagent storage depot can be a blister pack or other sealed chamber that can be ruptured on contact with a sharp implement.

The two contacting steps, of the porous membrane with the fluidic sample and with the fluid containing a reagent, can be performed sequentially or simultaneously. In the latter case, the sample and reagent could, in addition to contacting the membrane simultaneously, contact one another and bind, forming species not present in either fluid alone and which species then bind to capture molecules present on the assay membrane. The contacting with reagent can be repeated with an additional fluid containing an additional reagent, for as many times as may be required for single or multiple analyte detection.

The regulating step can comprise, for example, activation of a pneumatic device, a pump, or a gravitational force. A pump can be used, for example, to move fluid from the upstream end of the channel toward the downstream end. The actuation of fluid can be used to force downstream air toward a waste channel, hydrophobic membrane or other region downstream of the porous membrane. One example of a pump is a syringe or other device having a plunger and capable of displacing fluid.

The pneumatic device can be used to apply pressure or a vacuum. In some embodiments, the application of an external force comprises applying a pressure of about 0.05 to about 10 psi within the first channel. The optimal pressure to be applied will vary with the fluid in use, the fluidic circuit of the particular assay device and other factors that also affect flow rates through the porous membrane and resistance to flow in the fluidic circuit (e.g., membrane pore size and membrane area; desired contact time). Accordingly, pressures of 1, 2, 3, 4, 5 psi are also contemplated for use with the methods of the invention as well as pressures between 0 and 1 psi. In other embodiments, the regulating step comprises removing air from the first channel. Accordingly, negative pressures can also be used to remove air, such as by vacuum or gated exposure to a region of reduced pressure. The air can be removed via passage through a hydrophobic membrane disposed in the first channel and/or via passage through a waste channel in communication with the first channel. Typically, positive pressure is applied upstream of the porous membrane, while negative pressure is applied downstream of the porous membrane, to move air downstream of the porous membrane.

In some embodiments, a valve is used to regulate fluid flow across the porous membrane. A valve can be used, for example, to provide gated communication between the first channel and a waste channel or other region of differing pressure relative to the first channel. In some embodiments, the valve requires activation of an external force to regulate its position between an open and a closed state. Such external forces can include gravitational force, air pressure and fluidic actuation. One or more valves can be incorporated into an assay device of the invention to alter the resistance in the fluidic circuit and regulate fluid flow.

The transverse flow of fluid across the porous membrane can be in either or both directions. For example, while sample and reagents may typically be directed from the upstream to the downstream direction through the channel, and accordingly, fluid flow is directed from the upper surface to the lower surface of the porous membrane, one can also direct fluid from the lower surface to the upper surface in a regulated manner. This reverse, transverse flow can be used to prolong exposure of the porous membrane to a fluid that has already been directed from the upper surface to the lower surface by bringing the fluid back up from the lower surface to the upper surface. In some embodiments, the reverse, transverse flow of fluid can be used to direct a fluid introduced from the second channel toward the first channel. This can be used for fluid containing analyte, reagent and/or buffer or other wash fluid.

Representative fluid samples for use with the invention comprise blood or its components (e.g., plasma, serum), urine, saliva or other bodily fluid. The method can further include wash steps, as appropriate, including the use of the regulating steps to control fluid flow across the porous membrane to facilitate the wash step(s).

The invention additionally provides a method of removing air from the first channel of the assay device. The method comprise applying an external force to the first channel whereby air present in the first channel is directed to a waste channel in communication with the first channel. In some embodiments, applying an external force comprises applying a vacuum to a waste channel in communication with the first channel whereby air present in the first channel is directed to the waste channel. In other embodiments, applying an external force comprises pumping fluid into the first channel whereby air present in the first channel is directed to a waste channel in communication with the first channel. In yet other embodiments, applying an external force comprises pressing air into the first channel whereby fluid in the channel displaces air present in the first channel, directing it to a waste channel in communication with the first channel. The waste channel can be upstream or downstream of the porous membrane. In some embodiments, a hydrophobic membrane is positioned between the waste channel and the first channel.

The reagents used in the methods of the invention are typically capture agents and/or detection reagents. In a typical embodiment, the capture agents and the detection reagents comprise antibodies and/or antigens. In some embodiments, the method further comprises delivering to the porous membrane an amplification reagent that binds to the detection reagents. The detection reagents are labeled, either directly or indirectly, and the detectable signal can be provided or amplified using known techniques and materials.

Detection of signal can be achieved by a variety of means known in the art, including but not limited to, measuring an optical property such as optical absorbance, reflectivity, optical transmission, chemiluminescence or fluorescence. In some embodiments, signal can be detected by eye. Optical readers are preferred when a quantitative measurement is desired.

### BRIEF DESCRIPTION OF THE FIGURES

FIGS. 1A-1C are schematic illustrations of a card design for a flow-through-membrane assay. Individual fluids 6 delivered to the membrane 24 from a pump reservoir 4 are likely to be separated from one another by air 8. The image in FIG. 1A shows two options for venting the air 8 upstream of the membrane 24 (upper right) and venting the air 8 downstream of the membrane 24 (lower right). In the upstream method, vacuum 22 may be applied continuously (FIG. 1B) or it may be vented to atmosphere. The downstream method is described schematically in FIG. 1C, in which a hydrophobic porous membrane 20 provides a vent for air 8, but does not allow liquid 6 to flow up to a specified pressure. The air vent 20 allows repeated delivery of different liquid reagents 6, separated by air 8, to the flow through membrane 24.

FIG. 2A (top view) and FIG. 2B (side view or cross-section) schematically illustrates an example of an overall card design used to meter and push reagents 6 across an assay membrane 24. Sample is delivered from reservoir 14 via valve V2 28. Labels of “L” are pressure 10 (+) or vacuum 22 (−) lines, and labels of “V” are on-card valves 28 that can be open (O) or closed (C) as indicated in FIG. 2C. Vent 2 vents to atmosphere.

FIG. 3 illustrates an exemplary method for venting air downstream of the assay membrane 24. The upper figure identifies fluid 6 positions in the channels 16, 26 at various action steps listed in the table at bottom. V1 (optional) and V2 are valves 28, and they are vented to atmosphere.

FIG. 4A is a cross-section and close-up schematic of the flow-through membrane assay format (not to scale), showing the dry reagent storage on porous pad 40 and flow-through assay membrane 24. Expanded view illustrates steps 1, 2 and 3, in which (1) capture molecule IgM is immobilized on membrane 24 and non-specific binding is blocked with BSA; (2) sample containing analyte (PfHRP2) is added and unbound sample is removed via wash; regions lacking capture molecules do not bind analyte; and (3) labeling conjugate (Gold-IgG) is added, followed by wash to remove excess label; only regions with capture molecules are then labeled.

FIG. 4B illustrates the design and image of an assembled 10-layer assay card 46, showing the assay membrane 24, channel 38 through the membrane 24, sample loop 16, conjugate pad 40, bubble venting line 42, and waste line 44. The inset image shows the pattern of capture regions 48 visible on the membrane 24 after completion of the assay. The card 46 measures 83 by 52 mm, and is 2.3 mm thick.

FIG. 5A is a bar graph of relative PfHRP2 assay signal generated by different preservation formulations, depicting how assay performance is affected by the presence of sugar in the liquid-phase anti-PfHRP2 gold-antibody conjugate. Duplicate assays were run with samples containing 400, 200, 100, 50, 25, and 12.5 ng mL-1 of PfHRP2 in FBS. For each sugar loading, the chart plots the average and SD of the blank-subtracted signals obtained for these six antigen concentrations, relative to that of the no-sugar-added conjugate. A decrease in signal strength with increased sugar loading is evident.

FIG. 5B is a comparison of signal preservation after 60 days of storage, and shows the effect of long-term dry storage of the conjugate on assay performance. The chart shows the highest blank-subtracted signal obtained (n=3) over the duration of a 60-day study (white bars), compared to that obtained on day 60 (black bars). Four sugar loadings and 3 storage temperatures were compared, and assays were run on days 1-4, 6, 8, 12, 16, 42, and 60. All sugar loadings are given in weight/volume percentages. The earliest assay signals (days 1-3) were lower than subsequent measurements due to improvements in the capture surface over the first few days. This effect was subsequently reproduced, and is believed to be related to increasing stability of the antibody-nitrocellulose binding as the membrane dries in low-humidity storage. In order to avoid misinterpretation caused by a comparison with day-1 assay signals, the day-60 signals are compared to the highest signals observed over the study duration.

FIG. 6A is a diagram of channel geometry and fluid flow for reconstituting reagent dried on conjugate pads 40 in either lateral (upper portion) or transverse (lower portion) flow geometries. On the left, a schematic of the desired flow lines is pictured. On the right, the channel geometries 50 and observed flow are pictured. The transverse-flow geometry did not perform as desired, and the dashed lines indicate areas where air was not reliably displaced by fluid. The wicking action of the pad 40 caused fluid to enter the pad 40 through the first point of contact rather than through the whole top surface of the pad 40, and the large exit area below the pad 40 occasionally trapped air.

FIG. 6B is a series of images of reagent reconstitution and release from a conjugate pad. The pad measures 0.25 inches in diameter.

FIG. 6C is a plot of the conjugate pad release profile for a flow rate of 0.5 μL s⁻¹, based on 7 replicate measurements. The inset is an image of the channel downstream of the pad, with the advancing fluid front visible to the right of the image. The white box identifies the in-channel ROI in which the measurements were made. The dark debris on channel edges is adhesive and did not affect the release profile measurements.

FIG. 7A is five video frames from an on-card PfHRP2 assay. The images show the development of red spots at PfHRP2 capture antibody regions during the addition of gold-antibody conjugate. Nonuniform conjugate concentration is evident across the assay membrane.

FIG. 7B is a plot of assay response versus antigen concentration for 8 low-concentration samples of PfHRP2 in FBS (two at each concentration). The lowest non-zero concentration is 12.5 ng mL⁻¹ or 0.212 nM, and the assay response is as defined in Example 2 below. Intensities are from assay cards that have been run to completion.

FIG. 8A is an image of the microfluidic cards 46 developed to detect malarial proteins. Reagents are stored dried on-card. Noted are the volume metering chamber 80, dried Au labeling reagents 82, hydrophobic membrane 20, assay membrane 24, and air vent 2.

FIG. 8B illustrates the metering system for sample and reagent volumes 6. The hydrophobic membrane 20 passes air, but not fluid 6 at the pressures used. First, vacuum 22 draws fluid 6 from on-card reservoirs 14 toward the hydrophobic membrane 20 (upper). Fluid volume 6 is defined by the channel 16 dimensions (center). Third, pressure is applied, the valve 28 states changed, and the fluid 6 is driven through the card 46.

FIG. 9 is a schematic illustration of a section cut of the assay membrane 24 region of the microfluidic card 46. The fluid channel 16 approaches the membrane 24 from the right, expelling air 8 out via the hydrophobic membrane 20 to air vent 2, until the vent 2 is covered by fluid. Then the fluid is forced through the assay membrane 24, a much higher resistance path, then to waste 44. The “ledge” 90 traps bubbles 8, so the channel 16 geometry aligns this ledge 90 under the air vent 2 so the bubbles 8 are all removed and do not obstruct imaging of the assay membrane 24.

FIG. 10 is an image of the assay membrane from a 500 ng/mL sample of PfHRPII antigen spiked into human plasma (enhanced for improved printing). The spots are locations where Au secondary detection species were captured, indicating positive signal. The table shows the average normalized signal intensity found after imaging the completed assay. The normalized intensity was equal to 1−(average greyscale intensity/total greyscale levels). Pure white is now zero and pure black, one.

### DETAILED DESCRIPTION OF REPRESENTATIVE EMBODIMENTS

The invention relates to a microfluidic card incorporating a porous membrane for carrying out multi-step immunoassays. The membrane has a surface area about 300× larger than a flat surface; this greatly increases the sensitivity of measurement. The small membrane pores also lead to very rapid diffusion, even for large proteins. Slow diffusion is the cause of slow assays in conventional plate formats, and the membrane format virtually eliminates this limitation. The flow-through membrane microfluidic assay takes advantage of the high surface area and rapid transport but also allows individual control over flow rates and times for each step of the multi-step assay. Thus, the incubation step can be separately tuned for the sample, each reagent (e.g., secondary antibody, enzyme substrate), and each wash.

The system features include a microfluidic card with channels in communication with a porous membrane, channels on either side of membrane to allow transverse flow across the membrane, capturing a labeled target from the sample by flowing the sample across the membrane, or capturing a target from the sample followed by flowing a reagent containing a label that binds to the target. Fluid can be pushed or pulled through the assay membrane by external control (pumping, pressure, vacuum, gravity), thereby allowing different flow rates and times for each component. The invention further provides methods for managing air near the membrane. This can be accomplished by diverting air between fluids to a channel upstream of the assay membrane, venting air between fluids through a hydrophobic membrane upstream of the assay membrane, and/or by venting trapped air through a hydrophobic membrane downstream of the assay membrane. In some embodiments, the invention also provides storage and rehydration of reagents on porous carriers, and delivering the reagents by flow to the assay membrane.

In addition to the use of flow-through membranes for assays, the devices of the invention can be used for other microfluidic operations, including filtering, solid-phase assay, and selective capture. In various operations, including assays, it is often desirable to separate reagents by an air gap to prevent Taylor dispersion, other intermixing between reagents, density gradients, etc. A problem with some membranes is that once wetted, they do not allow air to flow through them at reasonable pressures and they often break. Various designs described herein provide a vent to allow air to escape between multiple fluid steps. In one approach, air is diverted between fluids to a channel upstream of the assay membrane. In another, air is vented between fluids through a hydrophobic membrane upstream of the assay membrane. In yet another embodiment, trapped air is vented through a hydrophobic membrane downstream of the assay membrane

### DEFINITIONS

All scientific and technical terms used in this application have meanings commonly used in the art unless otherwise specified. As used in this application, the following words or phrases have the meanings specified.

As used herein, a “channel” refers to a defined space through which fluid can travel. In a typical embodiment, the channel is defined by a plurality of walls, as well as an input, or upstream, end and an output, or downstream, end. Where a channel is defined by four walls, each perpendicular to its neighboring wall, the wall at the bottom of the channel is referred to as a “floor”, and the wall at the top of the channel is referred to as a “ceiling”. It is understood, however, that the invention is not limited to channels having a conventional shape (e.g., channels can have more or less than four walls, and a wall need not be perpendicular to its neighboring walls). Accordingly, the terms “floor” and “ceiling” are used as reference, such as to describe the relative positions of the assay membrane and the first and second channels, and are not intended to limit the channel configuration.

As used herein, “immobilized on the porous membrane” means immobilized on the upper and/or lower surface of the porous membrane, and/or throughout the membrane. Accordingly, an agent can be immobilized on the porous membrane without necessarily being on a surface of the membrane.

As used herein, “application of an external force” to regulate the flow of fluid means a force is applied to the device that modulates the flow of fluid by means other than the capillary action (surface tension) of the membrane. The force can be negative or positive pressure, a force generated by a pump or vacuum, or gravitational force, such as would affect the static head of the fluid.

As used herein, “valve” means a movable part that can be opened or closed. When opened, the valve allows fluid and/or gas to pass through and allows communication of pressure across the valve; when closed, the passage of fluid and/or gas is obstructed and the pressures on opposite sides of the valve are regulated independently of one another.

As used herein, a “plurality” means more than one of the indicated material. This can include more than one member of the indicated class of material, or more than one of the same member of the indicated class of material. For example, a plurality of reagents can refer to both heterogeneous and homogeneous populations of reagents.

As used herein, “a” or “an” means at least one, unless clearly indicated otherwise.

### EXAMPLES

The following examples are presented to illustrate the present invention and to assist one of ordinary skill in making and using the same. The examples are not intended in any way to otherwise limit the scope of the invention.

### Example 1

### Venting Air Away from Assay Membrane

Three approaches to venting air away from the assay membrane to facilitate regulated fluid flow across the membrane are summarized. One approach involves diverting air between fluids to a channel upstream of the assay membrane. This approach is described in greater detail in Example 5 below entitled “Enabling a microfluidic immunoassay for the developing world by integration of on-card dry-reagent storage”. See also “Air removal by waste channel upstream of the assay membrane” appended to this document. A second approach relates to venting air between fluids through a hydrophobic membrane upstream of the assay membrane, and a third to venting trapped air through a hydrophobic membrane downstream of the assay membrane.

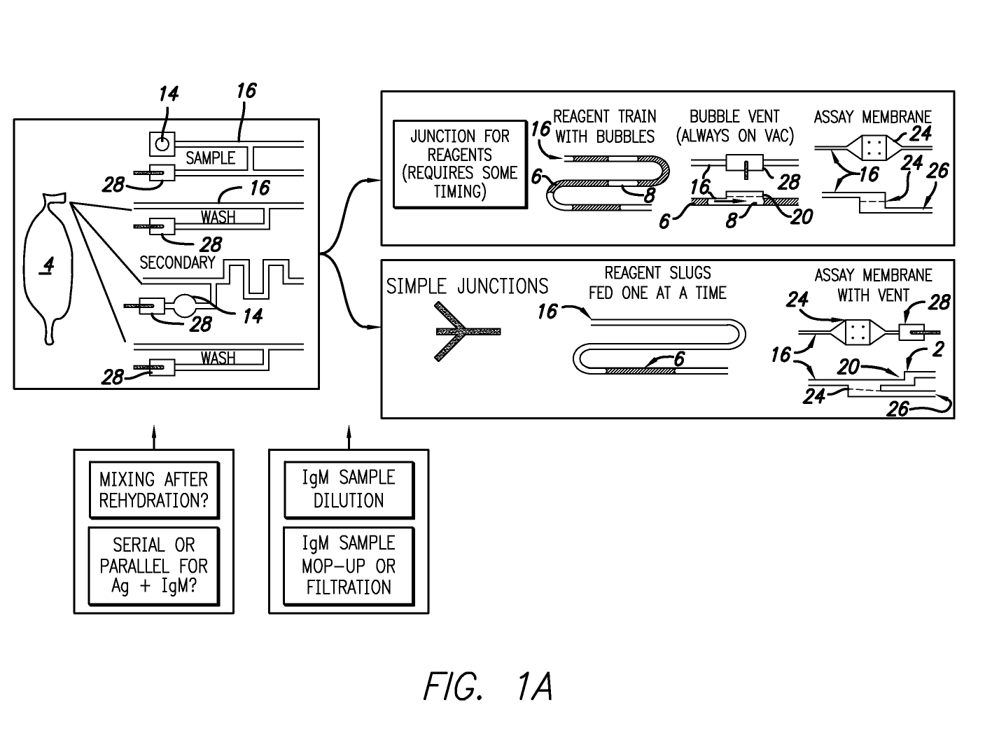

A general description of a card design for flow-through-membrane assay is illustrated in FIG. 1A. Individual fluids delivered to the membrane are likely to be separated from one another by air. Since the air cannot pass through a wet membrane, the air must be vented. The image shows two options for venting the air upstream of the membrane (upper right) and venting the air downstream of the membrane (lower right). In the upstream method, vacuum may be applied continuously or it may be vented to atmosphere.

The second (upstream) method is described schematically in FIG. 1B. This method is suited for any method of controlling fluid, and it has been tested successfully for cards using pressure and vacuum to move fluids. A hydrophobic porous membrane is used to extract air before the reagent reaches the membrane so that reagents are flowed through the membrane in a sequence but without air bubbles in between each reagent. A continuous train of reagents is thus stacked together without air gaps. A potential disadvantage of this method is the risk of reagent interdiffusion and reagent dispersion, such as Taylor dispersion (dispersion in the direction of flow due to the non-uniform velocity profile) and dispersion due to flow created by density differences between reagents. Placing the vent close to the assay membrane limits the distance that must be traveled by adjacent (front-to-back) reagents (reduces Taylor dispersion) and the time that it will take to transit the distance (reduces interdiffusion). In practice, some amount of dispersion or interdiffusion can be tolerated without significant impact on the assay.

In one test of this method, an on-card assay with dry reagents for testing for histidine rich protein-II (PfHRPII, an indicator of a P. falciparum malaria infection) was constructed. The card used 20 μl dry Au-secondary antibody, about 180 μl PfHRPII in FBS. The assay took less than 9 minutes. Flow rates were adjusted to improve assay signals. Lower flow rates and longer exposure times increased signal and provided more consistent distribution of reagent across the membrane. Further development and improvements of this assay are described in Example 2.

The same card design was used to test an on-card biplexed IgM assay for rickettsia and measles. Microflow syringe pumps were used, along with 20 μl dry anti-IgM Au (Arista), about 180 μl sample, and rickettsia and measales antigen at the assay membrane. The protocol was also the same as that used for PfHRPII, except for a 4 minute incubation (12 μl at 0.05 μl/sec) for Au-antibody instead of two minutes, making for 11 minutes to result in the automated assay. Spot intensity was rated on a qualitative scale and found at the site of the rickettsia capture antibody to be “clearly visible” for the rickettsia positive sample, “not visible” for the measles positive sample, and “barely visible” for the control sample of normal human plasma. Spot intensity at the site of the measles capture antibody was “barely visible” for the rickettsia positive sample, “strongly visible” for the measles positive sample, and “not visible” for the control sample.

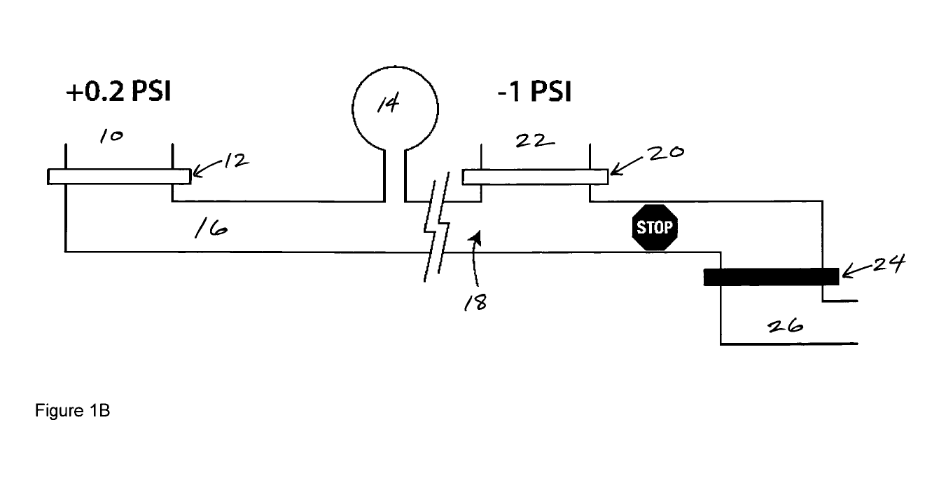

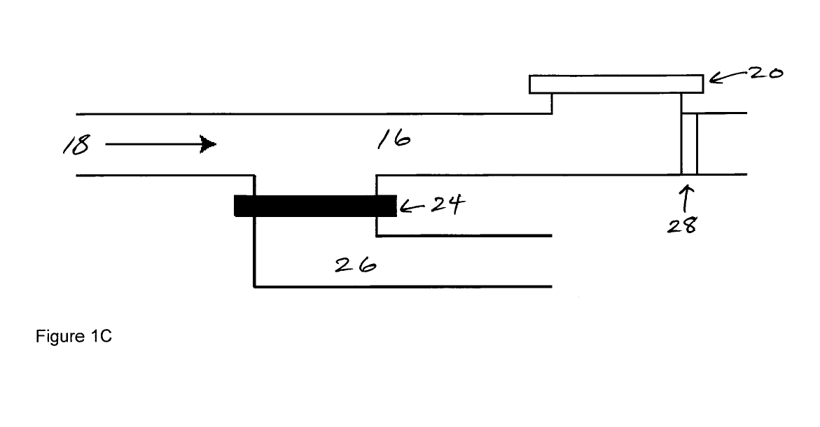

The third (downstream) method is described schematically in FIG. 1C, and in FIG. 2, and FIG. 3. A hydrophobic porous membrane (e.g., Mupor, Porex, Nomex) provides a vent for air, but does not allow liquid to flow up to a specified pressure. The air vent allows repeated delivery of different liquid reagents, separated by air, to the flow through membrane. FIG. 2A (top view) and FIG. 2B (side view or cross-section) schematically illustrates an example of an overall card design used to meter and push reagents across an assay membrane. Labels of “L” are pressure (+) or vacuum (−) lines, and labels of “V” are on-card valves that can be open (O) or closed (C) as indicated in FIG. 2C. “ATM” is a vent to atmosphere. Any number of reagents could be accommodated, and variations of pressure, vacuum, and atmosphere locations can be used and still preserve the basic function of venting air near the assay membrane. The valve V1 is used here only because the hydrophobic vent membrane is located away from the assay membrane. If they were co-localized, the valve would less important.

The hydrophobic membrane 20 allows trapped air to escape but does not allow fluid to escape. This membrane could simply be placed directly over the assay membrane and vented to atmosphere. In order to image the top of the assay membrane, the vent membrane 20 can be offset. The addition of a valve can be used to purge the small amount of liquid that would otherwise be trapped and foul the vent membrane. FIG. 3 shows an example set of steps driven by pneumatic control for an example assay. The valve V2 is not necessary—the vent membrane can be vented to atmosphere without valve control. The upper figure identifies ports used in the table at bottom. V1 and V2 are valves, and they are vented to atmosphere—V1 is not necessary. Each fluid component is in this example is controlled by opening valves that apply pressure to only the appropriate line. As the end of a fluid slug approaches the membrane, the rear fluid/air interface causes the flow to stop when it reaches the membrane—this occurs because moving beyond the membrane requires the fluid to contact more air than when it is in the membrane, and that is energetically unfavorable. Starting with description of 4a, applying pressure to the secondary reagent pushes air through the hydrophobic membrane—without this membrane, there would be no flow because air would be trapped. The secondary continues to flow until the rear interface stops on the membrane. The remaining liquid is vented by opening V2, and the process can be repeated many times for multi-step processes.

### Example 2

### Enabling a Microfluidic Immunoassay for the Developing World by Integration of on-Card Dry-Reagent Storage

This example describes a microfluidic flow-through membrane immunoassay with on-card dry reagent storage. By preserving reagent function, the storage and reconstitution of anhydrous reagents enables the devices to remain viable in challenging, unregulated environmental conditions. The assay takes place on a disposable laminate card containing both a porous membrane patterned with capture molecules and a fibrous pad containing an anhydrous analyte label. To conduct the assay, the card is placed in an external pumping and imaging instrument capable of delivering sample and rehydrated reagent to the assay membrane at controlled flow rates to generate quantitative results. Using the malarial antigen Plasmodium falciparum histidine-rich protein II (PfHRP2) as a model, this example demonstrates selection of dry storage conditions, characterization of reagent rehydration, and execution of an automated on-card assay. Gold-antibody conjugates dried in a variety of sugar matrices were shown to retain 80-96% of their activity after 60 days of storage at elevated temperatures, and the release profile of the reconstituted reagent was characterized under flow in microfluidic channels. The system gave a detection limit in the sub-nanomolar range in under nine minutes, showing the potential to expand into quantitative, multi-analyte analysis of human blood samples.

Improving global health requires accurate diagnostic technologies that are appropriate to the challenges of the developing world. In regions with limited health care systems, misdiagnosis may be especially costly, considering that treating the wrong disease wastes both meagre therapeutic budgets and limited time with health workers who may have only a single interaction to help patients in remote settings. Malaria, for instance, kills over a million people annually¹ and is subject to high rates of over-diagnosis in regions with large febrile populations.² Although effective methods of malarial diagnosis, such as the enzyme-linked immunosorbent assay (ELISA) and microscopy, are prevalent in well-equipped laboratories, assays in the developing world must couple accuracy and sensitivity with formats that accommodate challenges such as low diagnostic budgets, rough handling in remote locations, and a lack of refrigeration, regular power, and trained personnel.³⁻⁵

Microfluidic systems have a number of characteristics that may be brought to bear on these challenges, such as the ability to process and analyze small samples in an automated, rapid, and repeatable manner.^{6,7} Changes in scale have allowed improvements in sensitivity and detection limits,^{8,9} and some groups have been pursuing the use of inexpensive disposables as part of their methods for analyte detection or sample processing.^{10,11} Integration of reagent storage and result analysis on the microfluidic system reduces demands on end-users, thereby putting the entire analytical process within reach of lower-resource settings.

One approach to microfluidic systems couples a low-cost disposable assay card containing all necessary reagents with a portable reader capable of assay automation and quantitative optical measurement. This approach allows advanced control capabilities and a low cost per test, thus spanning the gap between sophisticated benchtop assays and disposable dipstick assays, the current standard for rapid diagnostic tests (RDTs) in the developing world.⁴

This example describes a rapid immunoassay format amenable to the disposable-and-reader model, using the malarial antigen Plasmodium falciparum histidine-rich protein II (PfHRP2) as an example. The assay is conducted on a laminate microfluidic card containing stable, anhydrous labeling reagent and a porous assay membrane, with external hardware handling the fluid pumping and optical readout. The assay is conducted in under nine minutes by injecting the sample into the card, clamping the card ports in a pump interface manifold, and starting an instrument script that rehydrates the on-card reagent and pushes the sample and labeling reagent through the assay membrane.

For developing-world applications, dry-form reagent storage is particularly important for its ability to preserve reagent function in environments with high local temperatures and a lack of refrigeration. Anhydrous on-card storage can also simplify assay automation, which can improve assay repeatability and reduce user training requirements. The dry storage method demonstrated in the PfHRP2 assay cards allows simple drop-in addition of reagent pads, as opposed to other methods that require fluid application during card assembly.^{13,14}

This assay system addresses some of the unmet needs of existing tests.³ The ELISA format provides a quantitative test for PfHRP2 with a low detection limit, but it requires manual operation, controlled storage conditions, and reagent incubation times on the order of hours. The lateral flow or immunochromatographic strip (ICS) assay format provides low-cost tests and simple operation, but it allows only simple fluidic manipulation, is limited in automation capability, and typically generates only qualitative results. Additionally, since most sandwich-assay ICS tests accommodate only a single assay step that mixes sample and secondary label prior to capture, they are subject to signal attenuation at high analyte concentrations in what is known as the “hook” effect.¹⁵ In contrast to these methods, the approach presented here is capable of sophisticated flow-rate control, sequential fluid addition for multi-step assays, multiplex formatting, complete automation, and result quantification in a low-cost disposable that withstands unrefrigerated storage.

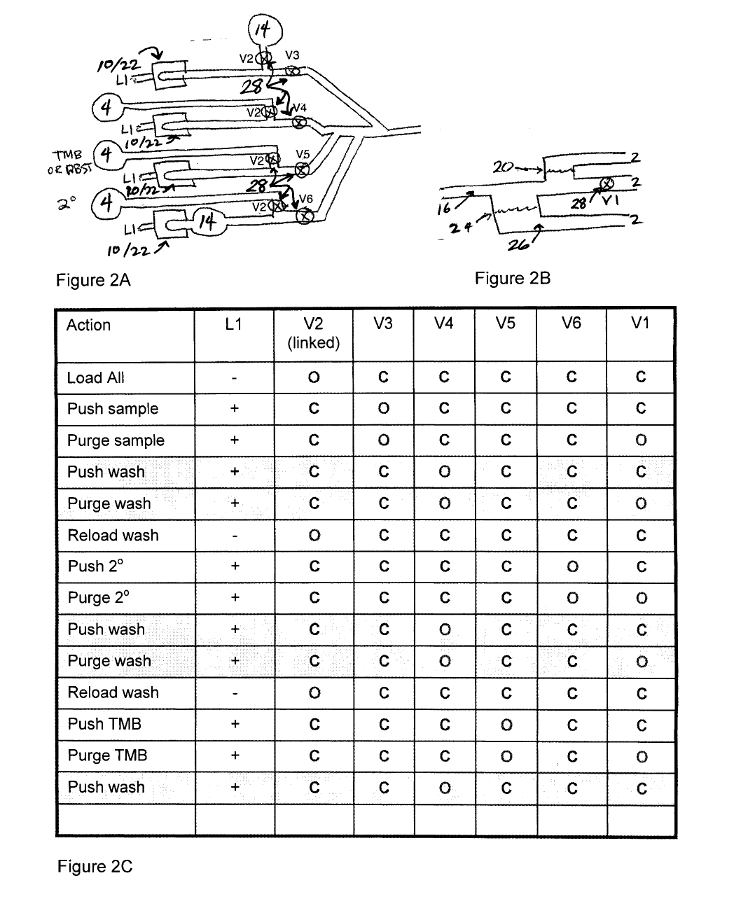

The central component of the assay is a laser-cut porous membrane, patterned with capture molecules and encased in a channel that directs fluid through the membrane. For a sandwich assay (FIG. 4A), a sample is passed through the membrane at a controlled flow rate by an external pumping mechanism, allowing capture of sample analyte, followed by a buffer wash to remove unbound sample. Buffer is also passed over a fibrous pad containing a labeling reagent dried in a preservative matrix. The rehydrated reagent is then passed through the assay membrane, allowing binding of the label to the captured analyte, followed by a wash to remove unbound label. For visible labels, the quantity of captured analyte can be measured by estimating the amount of bound label from a video or still image of the assay results. The example analyte used in this study is PfHRP2, a water-soluble protein produced by the Plasmodium falciparum strain of malarial parasites that induces heme polymerization in erythrocyte hosts.¹⁶⁻¹⁸ It is commonly used in RDTs as a plasma biomarker for schizogony, the parasite's asexual reproductive process that releases PfHRP2.¹⁹⁻²¹ In this study, PfHRP2 is added to and detected in fetal bovine serum, which acts as a complex but noninfectious matrix substituting for human plasma. Antibody bound to the assay membrane acts as the capture molecule and gold-antibody conjugate stored in a sugar-based matrix acts as the label, generating a visible increase in optical density proportional to the concentration of analyte present.

This system shares some materials with ICS tests: a porous assay membrane patterned with capture molecules and a fibrous pad containing dry gold-antibody conjugates in sugar. The porous membrane provides advantages over planar assay substrates, including decreased diffusion distances and increased surface areas for binding. These factors contribute to shorter assay times and increased signal strength compared to those seen for a flat capture surface. The fibrous conjugate pad provides large surfaces for rehydration, and the sugar matrix acts to stabilize protein structure and thus preserve function.²²

By reformatting these components into microfluidic channels, several aims are advanced. The transverse flow of reagents, actuated by external pumps rather than capillary action, allows more sophisticated control of reagent flow through the membrane. Sequential reagent addition, variable flow rates, and automated timing are all possible, and parallel assay multiplexing is achieved by spatially separating different capture molecules on the membrane. Conjugate pads placed in microfluidic channels allow pick-and-place addition of reagents to devices, and because the pads can fill the volume of a reconstitution channel, they can also provide a means for more uniform release of reagent across a channel cross-section.

### Device Design and Fabrication

The disposable assay card design (FIG. 4B) consists of a chamber holding the assay membrane, three upstream fluid lines (sample line, conjugate pad line, and bubble venting line) connecting to syringe pumps, and a downstream waste line. Three pumps are required in this case because the cards are valveless, although simpler fluid actuation systems have been proven to work.

Nitrocellulose membrane (Whatman® Protran®, 0.45 μm pore size) was sandwiched between two polymer layers and cut with a 25-watt CO₂ laser (M-360, Universal Laser Systems Inc., Scottsdale, Ariz., USA).²³ The membrane was placed on a mechanically actuated stage and 0.15 μL of 0.25 mg mL⁻¹ anti-PfHRP2 IgM (National Bioproducts Institute, Pinetown, South Africa) was patterned in each of 16 locations in a 4×4 grid, giving capture spots 120 μm in diameter. The membrane was then blocked for 30 minutes in Zymed® Membrane Blocking Solution, followed by drying at 20° C. and storage in a desiccator.

Cards were built from laser-cut layers of adhesive-backed Mylar and PMMA (Fraylock, Inc., San Carlos, Calif., USA), as practised in this lab and elsewhere for over 10 years.^{24,25} To prevent non-specific binding, channel surfaces were blocked by immersion in 10% bovine serum albumin (BSA) for 30 minutes, followed by rinsing with deionized water and baking at 35° C. for 30 minutes. During final assembly, the card encased the assay membrane and a conjugate pad containing 20 μL of anti-PfHRP2 gold conjugate at OD₅₂₄=10. The assay membrane sat in the pocket of a 0.004 inch-thick Mylar layer, held in place between two adhesive-backed Mylar layers that forced fluid flow through the membrane (FIG. 4A).

### On-Card Assay Procedure

A sample consisting of recombinant PfHRP2 (Immunology Consultants Laboratory, Newberg, Oreg., USA) in fetal bovine serum (FBS) was injected by pipette into the sample line of the assay card until the fluid front reached a fiducial mark on the card (approximately 185 μL of sample). The card was clamped into the manifold of a microFlow™ fluidics workstation (Micronics, Inc., Redmond, Wash., USA), which provided positive-displacement pumping for the assays via syringe pumps and software control. Phosphate-buffered saline (PBS) with 0.1% Tween® 20 (PBST) filled each pump reservoir and was used to push the sample, rehydrate the gold conjugate, and wash the membrane.

A script ran the assay in the following steps: (1) pumping PBST into the conjugate pad to initiate conjugate rehydration; (2) pumping PBST through the sample line to drive sample slowly through the membrane; (3) pumping PBST quickly through the sample line to wash away unbound sample from the membrane; (4) pumping PBST from the conjugate pad line while drawing negative pressure on the bubble vent line to remove air from between the rehydrated conjugate and the PBST filling the assay chamber: (5) pumping PBST through the conjugate pad to drive conjugate slowly through the membrane; (6) pumping PBST through the membrane to wash unbound conjugate from the area. A range of volumes and flow rates of reagents were tested.

In a test of the detection limit of the card, the membrane flow-through area was 7.6 mm² and the volumes and flow rates for the above numbered steps were as follows: (1) 19 μL @ 4.0 μL s⁻¹ (conjugate reconstitution), (2) 120 μL @ 0.5 μL s⁻¹ (sample through membrane), (3) 300 μL @ 4.0 μL s⁻¹ (wash). (4) 9 μL @ 4.0 μL s⁻¹ (conjugate advance and air removal), (5) 12 μL @ 0.1 μL s⁻¹ (conjugate through membrane), and (6) 180 μL @ 4.0 μL s⁻¹ (wash). Note that the early conjugate reconstitution prior to use allowed approximately 6 minutes for the reagent to dissolve during other assay steps. Studies during card development showed that this approach improved uniform conjugate delivery compared to reconstituting the reagent immediately before introduction to the membrane. In total, the assay steps took under 9 minutes from the start of step 1 to the end of step 6.

### Dry Reagent Storage and Microfluidic Release

Gold colloid was produced by reduction of gold chloride by sodium citrate,²⁶ with an absorbance of OD₅₂₄=1.25 and a size of roughly 40 nm confirmed by TEM. Anti-PfHRP2 IgG (National Bioproducts Institute, Pinetown, South Africa) was conjugated to the colloid using methods described previously.²⁷ After centrifugation at 6500×G, the conjugate pellet was resuspended in a Tris-buffered saline solution containing BSA and was filtered through a 0.45 μm cellulose acetate filter. A similar product was also obtained commercially, with a peak absorbance of OD₅₃₄=10.0 (BBInternational, Cardiff, United Kingdom).

The spunbonded polyester conjugate pad (6613, Ahlstrom, Holly Springs, Pa., USA) was laser-cut into circles 0.25 inches in diameter, and then was soaked for 30 minutes in an aqueous solution containing BSA. The pads were dried for 30 minutes at 35° C. and were stored in a desiccator until used.

For dry preservation, sucrose and trehalose were added to the OD₅₂₄=10 conjugate at up to 10% w/v each. The pads were placed into the wells of a 48-well plate, previously blocked with 10% BSA, and 10-30 μL of conjugate was pipetted onto each pad. The plates were baked in a 35° C. oven until the pads were completely dry (1-4 hours) and were then transferred to a desiccator until used. In tests of long-term stability, pads were sealed in polyethylene bags with a pouch of desiccant and stored at 4, 20, and 40° C. to mimic refrigerated, room-temperature, and elevated outdoor environments.

To test reagent release, the conjugate pad was sealed in a laminated microfluidic card (prepared in the same manner as the assay card) with a chamber that directed fluid flow through the pad. A syringe pump (V6, Kloehn Ltd., Las Vegas, Nev., USA) pushed PBST into the dry chamber from an inlet, and rehydrated reagent exited the card via an extended observation channel with a rectangular cross-section measuring 0.02×0.12 inches.

### Vacuum Manifold Assay Procedure

To run the assay in a high-throughput benchtop format, a 96-well-plate vacuum manifold (Bio-Dot® Microfiltration Apparatus, Bio-Rad, Hercules, Calif., USA) was used. It sandwiches a membrane and a silicone gasket between a bottomless 96-well plate and a base with a vacuum inlet. In this arrangement, reagents pipetted into the wells are exposed to the membrane as the vacuum draws them through the membrane pores at a flow rate controlled by the vacuum pressure and fluid viscosity. Fluid from each well passes through a membrane area of 7.8 mm².

Each well location on a sheet of membrane was functionalized with capture molecules by pipette-spotting 3 μL of anti-PfHRP2 IgM at 0.5 mg mL⁻¹. The membrane was dried for 20 minutes at room temperature prior to being blocked and stored as above. To rehydrate dry reagents, pads were placed in microcentrifuge tubes with enough PBST to bring them to a calculated OD₅₂₄ of 2.5, based on the volume of conjugate loaded onto the pad, and vortexed for 30 seconds.

To run the assay, the membrane was immersed in PBS and clamped into the manifold. The vacuum was set to 5 inches Hg, and the following steps were followed for each well. (Unless vacuum is off, each step ends after the well empties.) (1) Add 100 μL PBST; (2) with vacuum off, add 200 μL sample (prepared as above); (3) vacuum sample through for 4 seconds, turn off vacuum for 4 minutes, and then turn vacuum back on; (4) add 600 μL PBST, followed by 600 μL PBS; (5) with vacuum off, add conjugate; (6) repeat steps 3 and 4 for the conjugate and final wash.

The end result of the vacuum manifold assay is a membrane patterned with a grid of assay regions. The optical density of each region tends to be uniform across the middle of the spot with a darker ring around the perimeter; quantification of the signal uses the uniform region near the center of each spot.

### Image Capture and Analysis

Assay results were imaged with a flatbed scanner (ScanMaker i900, MicroTek International, Inc., Cerritos, Calif., USA) in 48-bit RGB at a resolution of 600 ppi (vacuum manifold format) or 2400 ppi (on-card format). Images of the vacuum manifold results were quantified in MATLAB® (The Mathworks™, Natick, Mass., USA) by (1) semi-automated selection of regions of interest (ROIs) containing a uniform center region of each assay spot, (2) creation of a histogram of green-channel pixel intensities for each assay ROI, (3) mild low-pass filtering of the histogram, and (4) report of the histogram mode. “Blank subtracted” signals are the difference between the signal obtained from a sample of interest and that of a “blank”—a sample containing no analyte. Images of the on-card results were quantified in ImageJ²⁸ by (1) manual selection of regions of interest inside and outside each visible spot, (2) measurement of the mean green-channel pixel intensity of each ROI, (3) calculation of the difference between the ROI means inside and outside of each spot, and (4) report of the mean of these differences for all spots present.‡ On-card assays were also captured on a low-cost USB camera (AM211 Dino-Lite, AnMo Electronics Corp., Hsinchu, Taiwan) capable of quantifying the assays in the same manner.

Microfluidic reagent release was imaged at a magnification of 1× on a Nikon SMZ1500 microscope with an Optronics DEI-750D camera (Optronics, Goleta, Calif., USA) capturing 10 frames per second in the green channel. Images were quantified by (1) creating an absorbance image of each frame by comparing it to an image of the channel with only PBST present; (2) selecting one ROI in the channel and another outside of it; (3) correcting the mean absorbance of each frame's in-channel ROI by subtracting that of the out-of-channel ROI, which should be constant over time; (4) reporting the corrected absorbance. The method was validated with a dilution series of gold conjugate that was also measured on a spectrophotometer, allowing camera absorbances (based on a broad green spectrum) to be converted to OD₅₃₄ measurements.

Results

### Dry Reagent Storage

Sucrose and trehalose have been implicated in the stabilization of proteins and membranes in organisms that undergo complete dehydration.²² Adding them to protein-based reagents has been shown to stabilize the reagents in dry form.^{13,14,29} Three off-card experiments were conducted to determine how effectively the sugars could preserve function of the assay's labeling reagent, a gold-antibody conjugate, under the assumption that the off-card dry storage of reagent is similar or identical to dry storage on a device. The first experiment tested the effect the sugars have on liquid reagent, without any drying process. The second tested how effective various sugar loadings were at preserving function after drying and rehydrating the reagent. The last tested the long-term stability of dry reagent using the better-performing sugar formulations.

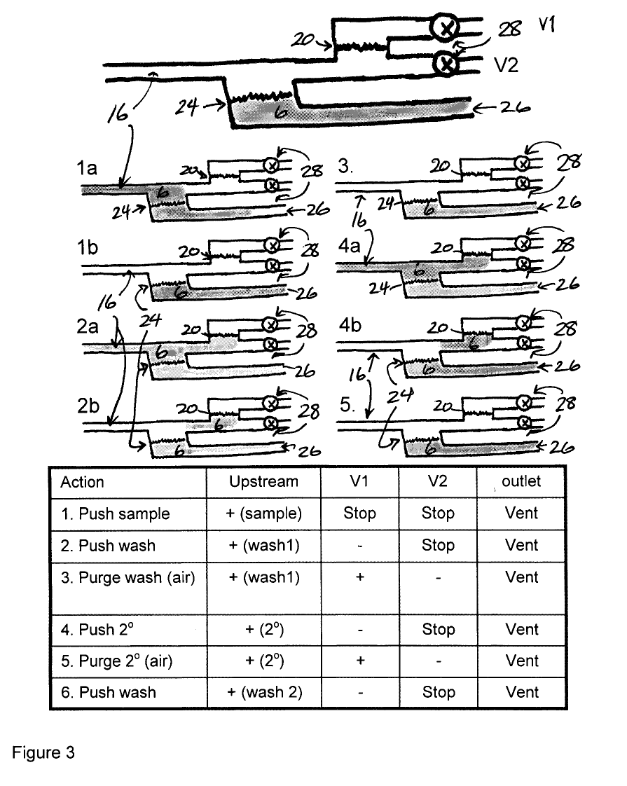

Adding sugars to liquid gold-antibody conjugate was shown to reduce assay signal strength in the vacuum manifold format (FIG. 5A). Using total sugar loadings of 0-15% w/v, it was found that both sucrose and trehalose interfered with signal production—higher sugar loading resulted in lower signals relative to an unloaded sample. Signal reduction ranged from 10-90%, and trehalose caused a greater decrease than sucrose. The reason for signal reduction is not clear, but the sugars may interfere with antibody binding or conjugate transport. Based on these results, lower sugar loadings would be preferred. In FIG. 5B, sugar-laden conjugate is shown to perform as well as the sugar-free conjugate, a result that appears to conflict with FIG. 2A. Note that the experimental conditions of these data differ: in FIG. 2A, the conjugates are tested in their original tris/BSA buffer, while in FIG. 2B, the sugar-laden conjugates have been dried and rehydrated in PBST. The apparently improved performance of the dried-then-rehydrated reagent is an unresolved issue that has appeared in other experiments and is being explored further in our group.

Initial attempts to preserve the conjugate reagent in a dry state demonstrated that sugar addition was required to preserve conjugate activity. Loadings of 0-10% sucrose and trehalose were added to aliquots of conjugate, and 50 μL of conjugate was added to each fibrous pad for drying. Assays were conducted using 160 μL of OD₅₂₄=2.5 conjugate per well in the vacuum manifold format. Samples without PfHRP2 (blanks) gave high non-specific signals at 0-5% total sugar loading, and gave low non-specific signals at 10% and higher loading. Samples with 200 ng mL⁻¹ PfHRP2 produced signals that were higher than the blanks—the magnitude of difference was consistent for sucrose/trehalose loadings of 5%/0%, 0%/5%, 5%/5%, and 10%/5%. A sugar loading of 5% sucrose and 10% trehalose, however, gave a lower signal difference from the blanks than the other formulations, likely due to the interference described above. Conjugate dried without sugars produced such a high non-specific signal that the 200 ng mL⁻¹ sample did not show a significant increase in specific signal. These results suggest that loadings greater than 5% are required to avoid non-specific signal production possibly caused by the formation of conjugate clusters that clog the membrane pores.

For long-term stability studies of the dry conjugate, sucrose/trehalose loadings of 5%/5%, 10%/5% and 5%/10% were chosen, and pads were loaded with 30 μL of OD₅₂₄=10 conjugate. Aliquots of liquid conjugate without sugar were stored alongside the dry conjugate at temperatures of 4, 20, and 40° C. In vacuum-manifold assays, long-term storage of dry gold-antibody conjugates showed preservation of 80-96% of signal after 60 days, compared to 6-55% for the liquid solution (FIG. 5B). These results indicate that the reagent should retain function on-card after long-term dry storage and rehydration in PBST. Preferred sugar formulations prevent rapid loss of reagent activity without interfering greatly with signal strength. For qualitative assays used shortly after production, lower sugar loadings may offer higher signal strengths. For quantitative assays used after longer storage periods, higher sugar loadings may offer greater signal stability.

### Microfluidic Reagent Reconstitution

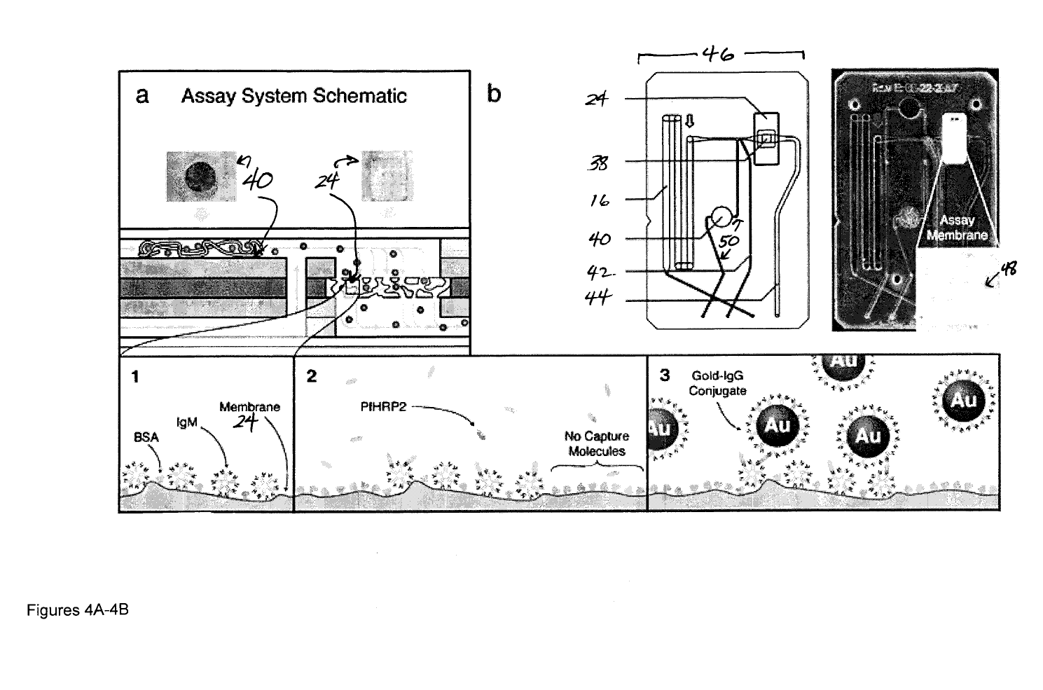

To be relevant to a microfluidic assay, the conjugate pads must be incorporated into on-card rehydration channels. Two channel designs were tested: one to push fluid laterally through the pad, and another to push it through in a transverse direction (FIG. 6A). It was believed that the two designs would give different reagent release profiles. In both designs, however, the pad's fibrous structure actively wicked fluid into the pad. When a fluid front reached the pad, the wicking action caused all buffer reaching the pad to enter it through the first point of contact rather than entering across the whole exposed pad surface. This result was observed for the range of flow rates tested (0.5-80 μL s⁻¹). This effect was observed on the large fluid-entry surface of the transverse-flow format—the entire top of the pad—resulting in an inconsistently performing design. The upper chamber remained mostly filled with air, while fluid exited the pad into the bottom chamber in an irreproducible manner. All subsequent rehydration designs sought to reduce the tendency of wicking to trap air in the channel, doing so by providing small inlets to the pad chamber that limited the potential contact area between incoming buffer and the pad. Sections of the pad closer to chamber edges are less-efficiently perfused, resulting in gradual removal of conjugate by a combination of slow convection and diffusion into faster-flowing streams. A geometry in which the pad width equals the constant channel width would likely release conjugate more consistently across its volume by providing more uniform perfusion to all areas of the pad.

Observation of the reconstituted conjugate 15 mm downstream of the pad showed a repeatable release profile. At a flow rate of 0.5 μL s⁻¹, rehydrated conjugate came out at a concentration of OD₅₃₄=25.6 (SD 4.1) and was clear of the pad and channel in 60-80 seconds (FIG. 6B-C). Some of the variation seen may be due to differences in pad loading. Although each pad received 20 μL of conjugate at OD₅₃₄=10, contact between the pad and the well in which it dried resulted in an inconsistent loss of conjugate from the pad. When seven pads were rehydrated in microcentrifuge tubes and their absorbance measured on a spectrophotometer, the absorbance CV was 9%. This variance could be lowered by changes to the pad loading technique. The repeatable reagent release profiles suggest that the rehydration technique can be used to deliver known concentrations of reagent to an assay surface over time.

### On-Card Immunoassay for PfHRP2

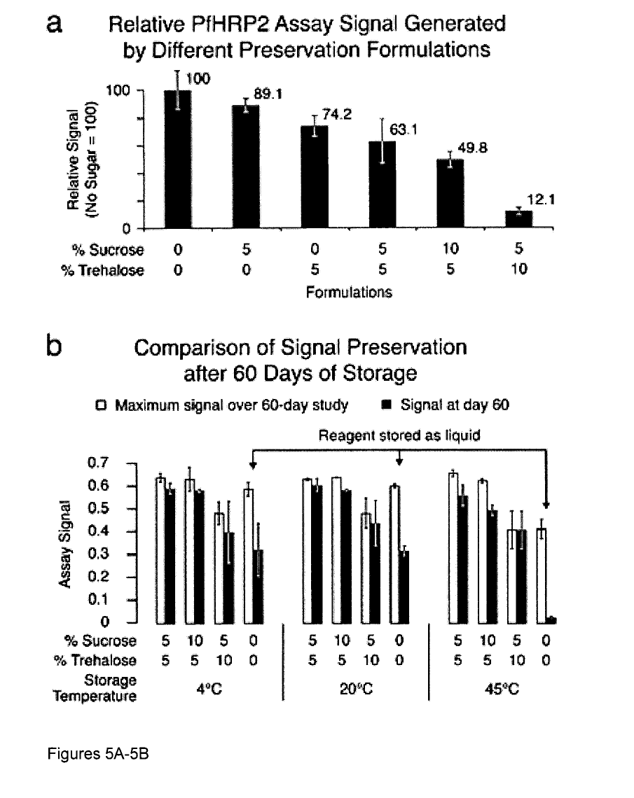

To produce an automated, rapid, and simple-to-use assay for PfHRP2, the assay was put on-card with an integrated dry reagent. The assay gave sub-nanomolar detection limits on the order of those obtained in a well-based ELISA assay for PfHRP2,³⁰ using an automated protocol completed in under 9 minutes (FIG. 7A-B). These results were obtained using conjugate pads and antibody-patterned membranes prepared and stored at room temperature for 3-4 weeks prior to card assembly and 30 days prior to the experiment, demonstrating potential for the long-term efficacy of the devices.

Two factors that affect the assay signal strength are the flow rate and the volume of reagent exposed to the membrane (for a set sample size, this is determined by the membrane's flow-through area). Lower flow rates gave higher assay signals, likely due to increased binding during a longer period of reagent exposure at the membrane. A membrane reduced from 140 mm² to 7.6 mm² in area gave an 18-fold increase in the volume of sample exposed to a given region of membrane. With a set fluid flux, this increase corresponds to an equal increase in the time the reagent is exposed to the membrane, which again resulted in a higher assay signal. These results suggest that attempts to decrease assay time need to be balanced with appropriate interaction times for reagent transport and binding. The porous nature of the assay membrane favors shorter assay times than planar substrates in this regard: shorter diffusion requirements should allow more rapid transport of analyte and conjugate to the membrane surface. The sample and conjugate are likely to require different flow rates as demanded by the diffusion rates of their components, and current work is focused on optimizing flow rates and volumes to maximize binding and signal production.

Other observations suggest opportunities for improving assay consistency. The dry reagent release profile of FIG. 6C predicts concentrations downstream of the conjugate pad when the rehydrated reagent displaces air, but in the current assay card the conjugate displaces PBST. Because the resuspended conjugate contains high concentrations of sucrose and trehalose, its density is greater than the PBST it displaces, thereby resulting in a gravity-induced segregation of the two fluids. If the fluids aren't sufficiently mixed before reaching the membrane, the nonuniform distribution of conjugate across the vertical dimension of the channel results in the far edge of the membrane receiving insufficient conjugate to produce an assay signal. A protocol-based fix for this problem was rehydrating the conjugate at the beginning of the assay, several minutes prior to use, and then passing it through the downstream PBST slowly. This change resulted in a weaker vertical gradient of conjugate concentration, but some reagent nonuniformity issues remained. As can be seen in FIG. 7A, parabolic flow profiles and Taylor dispersion focus conjugate in a plume down the center of the membrane. Pushing plugs of reagent with air bubbles should allow dispersion-free flow and improved uniformity of membrane exposure, although this approach will also require bubble removal prior to fluid reaching the membrane. Ongoing studies suggest that this approach is a feasible solution to these fluid delivery issues.

Lastly, many improvements remain to be made in aspects of the system that are not amenable to the final point-of-care setting for this assay. For instance, although serum-based samples were loaded by pipette in this study, this approach is not feasible in under-resourced settings. Finding appropriate methods for loading small volumes of whole blood, collecting the separated plasma, and metering the plasma sample for use in the assay can be adapted by those skilled in the art for global-health applications.

### CONCLUSIONS

The flow-through membrane immunoassay for PfHRP2 demonstrates a general approach to rapid, automated, quantitative assays that are appropriate to the challenges of point-of-care diagnostics. Adjustable fluid delivery capabilities allow the assay operation to be tailored to the particular flow requirements and protocols demanded by different assay cards. This approach may give more flexible and robust performance compared to those dependent on capillary action, which lack the ability to actively control flow rates and are susceptible to changes in the wetability of the wicking materials over time.³¹ Integration of on-card anhydrous reagent enables device storage in unrefrigerated environments, using a pad-based method that disperses reagent through the cross-section of the channel and generates repeatable release profiles in microfluidic channels. While other dry reagent methods have deposited liquid reagents into chambers or depots that require mid-assembly drying of reagents,^{13,14} the approach presented here allows simple drop-in inclusion of dry reagents at the time of assembly. Dry reagent pads can be prepared in bulk separately from the devices themselves, to be added by assembly when appropriate. The colorimetric assay results produced by this system can be quantified by low-cost cameras to estimate analyte concentration as an indicator of infection intensity.

The system described forms the groundwork for a more sophisticated and capable diagnostic tool, the advancement of which involves ongoing improvements in areas such as the following. Assay multiplexing is enabled by patterning multiple capture molecules in discrete regions across the membrane. Pneumatic pumping and valving allow simplification of on-card fluid actuation and plug-like flow that should improve reagent nonuniformities across the assay membrane. On-card fluid metering results in more consistent fluid volumes than the user-dependent approach described here. Fully-automated analysis of assay images allows objective quantification of assay results, and modifications to the analysis method should give a more linear signal-analyte relationship at high analyte concentrations. Leveraging the fluidic flexibility of the assay system, multi-step ELISA assays can be conducted on-card using a dried enzyme conjugate and liquid substrate. Progress has been made in all of these areas, the result of which is a new generation of device that is currently being tested. When combined with upstream blood separation and on-card storage of rehydration buffer, the system will be capable of sample-to-result quantification of multiple analytes from a human blood sample.

### REFERENCES

1. World Health Organization. World malaria report 2005, World Health Organization report WHO/HTM/MAL/2005.1102, Geneva, 2005.
2. H. Reyburn, et al., Br. Med. J., 2004, 329, 1212.
3. P. Yager, et al., Annu. Rev. Biomed. Eng., 2008, 10, 38.
4. P. Yager, et al., Nature, 2006, 442, 412-418.
5. C. D. Chin, et al., Lab Chip, 2007, 7, 41-57.
6. P. A. Auroux, et al., Anal. Chem., 2002, 74, 2637-2652.
7. G. M. Whitesides, Nature, 2006, 442, 368-373.
8. T. P. Burg, et al., Nature, 2007, 446, 1066-1069.
9. N. Christodoulides, et al., Lab Chip, 2005, 5, 261-269.
10. S. K. Sia, et al., Angew. Chem., Int. Ed., 2004, 43, 498-502.
11. X. Cheng, et al., Lab Chip, 2007, 7, 170-178.
12. A Point of Care Diagnostic System for the Developing World, http://www.gcgh.org/MeasureHealthStatus/Challenges/DiagnosticTools/Pages/PointofCare.aspx, accessed Jun. 30, 2008.
13. L. G. Puckett, et al., Anal. Chem., 2004, 76, 7263-7268.
14. E. Garcia, et al., Lab Chip, 2004, 4, 78-82.
15. S. A. Fernando and G. S. Wilson, J. Immunol. Methods, 1992, 151, 47-66.
16. R. J. Howard, et al., J. Cell Biol., 1986, 103, 1269-1277.
17. N. T. Huy, et al., J. Biochem., 2003, 133, 693-698.
18. V. Papalexis, et al., Mol. Biochem. Parasitol., 2001, 115, 77-86.
19. V. Desakorn, et al., Trans. R. Soc. Trop. Med. Hyg., 2005, 99, 517-524.
20. I. Clark, PLoS Med, 2006, 3, e68, author reply e69.
21. C. Wongsrichanalai, et al., Am. J. Trop. Med. Hyg., 2007, 77, 119-127.
22. J. H. Crowe, et al., Annu. Rev. Physiol, 1998, 60, 73-103.
23. P. Spicar-Mihalic, et al., Micro TAS Proc., 2007, 667-669.
24. A. Hatch, et al., Nat. Biotechnol., 2001, 19, 461-465.
25. B. H. Weigl, et al., Adv. Drug Delivery Rev., 2003, 55, 349-377.
26. G. Frens, Nat. Phys. Sci., 1973, 241, 20-22.
27. C. De Roe, et al., J. Histochem. Cytochem., 1987, 35, 1191-1198.
28. W. S. Rasband, ImageJ, U.S. National Institutes of Health, Bethesda, Md., USA, http://rsb.info.nih.gov/ij/, 1997-2007.
29. S. Ramachandran, et al., Proceedings of the 1st Distributed Diagnosis and Home Healthcare (D2H2) Conference, 2006, pp. 16-19.
30. C. M. Kifude, et al., Clin. Vaccine Immunol., 2008, 15, 1012-1018.
31. S. Haeberle and R. Zengerle, Lab Chip, 2007, 7, 1094-1110.

### Example 3

### Rapid Air-Driven Point-of-Care Malaria Detection

This example demonstrates pneumatically actuated microfluidic cards that provide an inexpensive multiplexable platform for the point-of-care (POC) detection of disease, exemplified here for malaria (P. falciparum), in under nine minutes. Reagent volumes are metered and sequentially driven through a porous membrane used as a flow-through substrate for a sandwich immunoassay (SIA). An initial test of 500 ng/mL PfHRPII spiked into human plasma produced signal intensity six times greater than the local background. This successful test demonstrates the conversion of a multi-step benchtop immunoassay into a fully-automated microfluidic format while retaining the potential to be quantitative.

These microfluidic cards use a novel flow-through membrane format controlled by a fully automated, pneumatically driven system. The SIA is performed on the surface of a porous membrane that the reagents flow through. The small pores decrease diffusion distances, which shortens assay time. The high surface area increases the available capture surface, potentially increasing signal intensity.

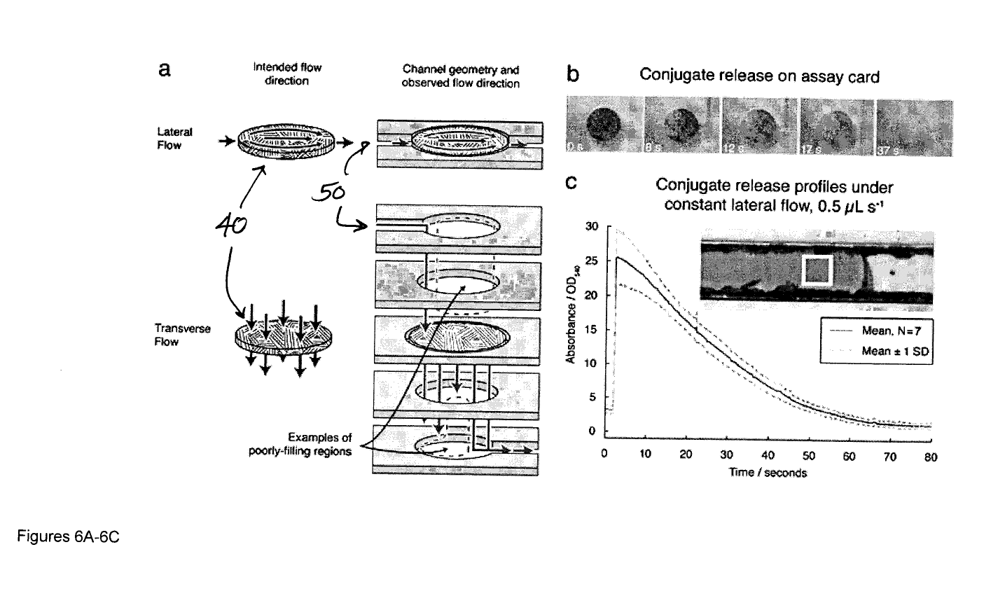

The capture antibody (0.25 μg/mL anti-PfHRPII IgM) is immobilized on the membrane, then blocked and dried before integration into the cards. Samples are PfHRPII spiked into human plasma, and Au-labeled secondary detection antibodies are stored dried on-card, increasing the simplicity of operation and potential storage time (FIG. 8A). The assay membrane is rinsed with buffer between each reagent.

A pneumatic pumping system was chosen by the collaborative team. Pneumatic systems can be rugged and less expensive than a system of syringe pumps. However, pneumatic systems apply constant pressure or vacuum, but the volumetric flow is determined by channel dimensions. Our cards use a system of volume metering reservoirs that terminate at air-permeable membranes to deliver reproducible reagent volumes to the assay membrane (FIG. 8B).

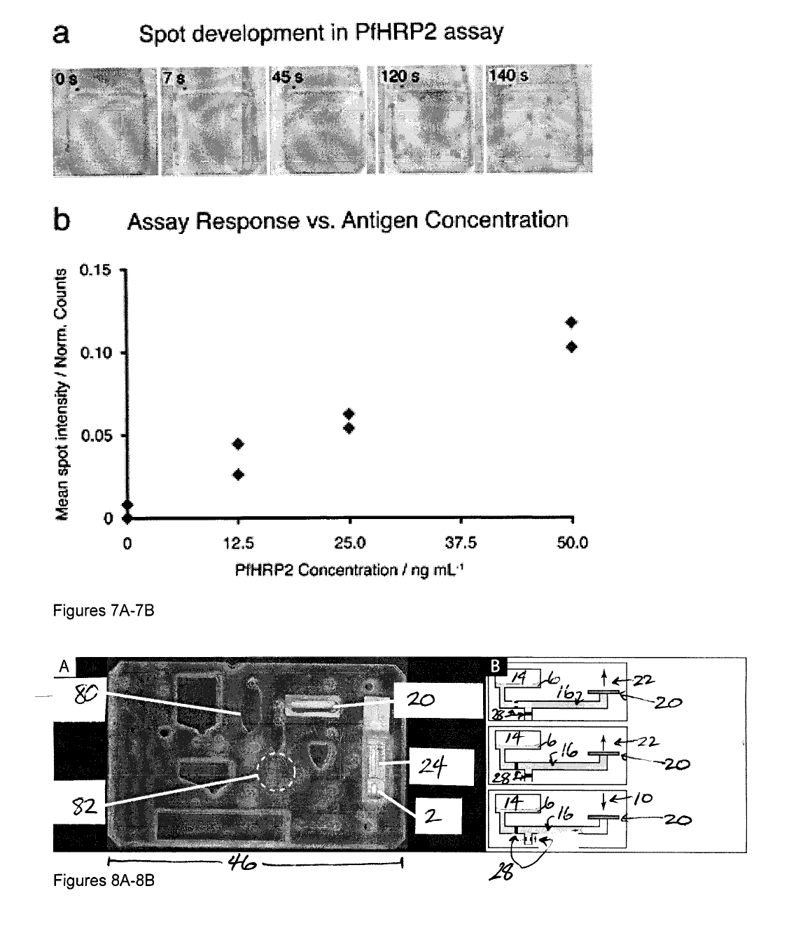

The assay membrane does not pass air after wetting, so the air from the sequential reagent deliveries is vented through another hydrophobic membrane. The microfluidic cards were fabricated from laser-cut laminate layers; the assay membrane was sandwiched between layers to secure and seal it in place. The incoming fluids experienced an increase in channel height at the transition onto the assay membrane. The second transition back to a narrower channel, or “ledge”, was arranged under the hydrophobic vent to minimize bubble formation resulting from that transition (FIG. 9).

### Results

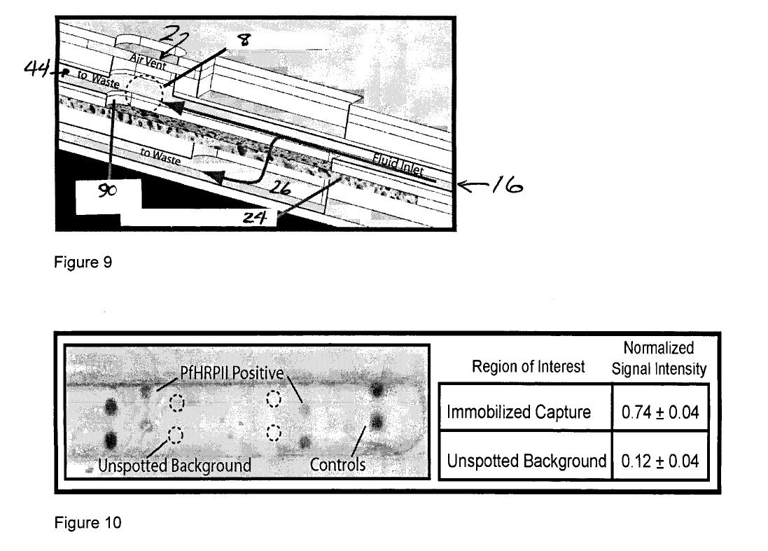

These microfluidic devices were validated by detecting PfHRPII proteins spiked in human plasma (FIG. 10). The signal intensity produced by 500 ng/mL PfHRPII sample was more than six times the signal of the unspotted background regions. A similar immunoassay, using syringe flow, has been shown to be quantitative.² Design robustness was confirmed: during initial testing, every device (n=13) demonstrated the expected reagent delivery to the membrane, and there was no bubble interference.

### REFERENCES

[1] Yager, P., et al., Nature, pp. 442, 412-418, (2006).
[2] Stevens, D. Y., et al., Lab on a Chip, pp. 2038-2045, (2008).

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An assay device for detection of an analyte in a fluidic sample, the device comprising:
(a) a microfluidic chamber having:
(i) a first channel defined by walls and a floor, the first channel having an upstream end and a downstream end, wherein fluid brought into contact with the first channel flows from the upstream end toward the downstream end; and wherein the floor comprises a region between the upstream and downstream ends that contains a porous membrane having an upper surface and a lower surface;
(ii) a second channel defined by walls and a ceiling, wherein the ceiling comprises the lower surface of the porous membrane;
(iii) one or more capture agents immobilized on the porous membrane;
(b) means for regulating the flow of fluid transversely through the porous membrane across the upper surface and the lower surface via application of an external force within the first and/or second channel.

2. The assay device of claim 1, wherein the means for regulating the flow of fluid comprises a pneumatic device, a pump, a valve, or altering the static head of fluid in the first channel.

3. The assay device of claim 2, wherein the pneumatic device comprises a pump or a vacuum.

4. The assay device of claim 1, further comprising a hydrophobic membrane disposed within the first channel.

5. The assay device of claim 1, further comprising a reagent storage depot in communication with the first channel, and one or more detection reagents disposed within the storage depot.

6. The assay device of claim 5, wherein the reagent storage depot comprises a porous material.

7. The assay device of claim 5, wherein the reagent storage depot comprises a sealed chamber that releases the detection reagents into the first channel upon rupture of the sealed chamber.

8. The assay device of claim 1, further comprising means for detecting analyte bound to the capture agent on the porous membrane.

9. A method for detection of an analyte in a fluidic sample, the method comprising:
(a) contacting the fluidic sample with the porous membrane of the assay device of claim 1;
(b) contacting a fluid containing a reagent with the porous membrane;
(c) regulating the flow of fluid transversely through the porous membrane across the upper surface and the lower surface via application of an external force within the first and/or second channel; and
(d) detecting the presence of analyte bound to reagent on the porous membrane.

10. The method of claim 9, wherein the contacting of step (b) comprises contacting a fluid with a reagent storage depot disposed within the assay device, wherein the reagent is stored in the storage depot in anhydrous form and is mobilized upon contact with the fluid.

11. The method of claim 9, wherein the contacting of step (b) comprises rupturing a reagent storage depot disposed within the assay device, wherein the reagent is stored in the storage depot and is mobilized upon rupture of the reagent storage depot.

12. The method of claim 9, wherein the contacting of steps (a) and (b) occurs sequentially.

13. The method of claim 9, wherein the contacting of steps (a) and (b) occurs simultaneously.

14. The method of claim 9, wherein the contacting of step (b) is repeated with an additional fluid containing an additional reagent.

15. The method of claim 9, wherein the regulating of step (c) comprises activation of a pneumatic device, a pump, or a gravitational force.

16. The method of claim 9, wherein the pneumatic device applies pressure.

17. The method of claim 9, wherein the pneumatic device applies a vacuum.

18. The method of claim 9, wherein the application of an external force comprises applying a pressure of about 0.05 to about 10 psi within the first channel.

19. The method of claim 9, wherein the regulating of step (c) comprises removing air from the first channel.

20. The method of claim 19, wherein the air is removed via passage through a hydrophobic membrane disposed in the first channel.

21. The method of claim 19, wherein the air is removed via passage through a waste channel in communication with the first channel.

22. The method of claim 9, wherein the fluid sample comprises blood, urine, saliva or other bodily fluid.

23. A method of removing air from the first channel of the device of claim 1, the method comprising applying an external force to the first channel whereby fluid in the first channel displaces air present in the first channel, directing the air to a waste channel in communication with the first channel.

24. The method of claim 23, wherein applying an external force comprises applying a vacuum to a waste channel in communication with the first channel whereby air present in the first channel is directed to the waste channel.

25. The method of claim 23, wherein applying an external force comprises pumping fluid into the first channel whereby air present in the first channel is directed to a waste channel in communication with the first channel.

26. The method of claim 23, wherein applying an external force comprises pressing air into the first channel whereby fluid in the first channel displaces air present in the first channel, directing the air to a waste channel in communication with the first channel.

27. The method of claim 23, wherein the waste channel is upstream of the porous membrane.

28. The method of claim 23, wherein the waste channel is downstream of the porous membrane.

29. The method of claim 23, wherein a hydrophobic membrane is positioned between the waste channel and the first channel.
